(19) United States
(12) Patent Application Publication
(10) Pub. No.: US 2018/0263516 A1
(43) Pub. Date: Sep. 20, 2018

(54) A DETECTION DEVICE FOR USE IN A BLOOD PRESSURE MEASUREMENT SYSTEM

(71) Applicant: KONINKLIJKE PHILIPS N.V., EINDHOVEN (NL)

(72) Inventors: Dieter WOEHRLE, WAIBLINGEN (DE); Norbert Ernst WUNDERLE, SINDELFINGEN (DE)

(21) Appl. No.: 15/761,822

(22) PCT Filed: Oct. 7, 2016
(86) PCT No.: PCT/EP2016/074014

§ 371 Date: Mar. 21, 2018

(30) Foreign Application Priority Data
Oct. 8, 2015 (EP) 15188876.5

(51) Int. Cl. A61B 5/022; A61B 5/00

(52) U.S. Cl. CPC A61B 5/02225; A61B 2560/0475; A61B 2560/0276; A61B 5/7221

(57) ABSTRACT

Disclosed is a detection device suitable for use with a blood pressure measurement system for detecting improper functioning due to the absence of an inflatable cuff from a patient's measurement site, its improper attachment to the site, or it being disconnected from the system. The device is coupled to an inflatable cuff attached to the measurement site and arranged to perform a series of measurements, wherein for each measurement of the series, an inflation operation is performed to inflate the cuff. The device includes a determining unit for determining inflation speed-dependent parameter values during inflation operations, and a processor module arranged for receiving the determined inflation speed-dependent parameter values. The processor module is configured to check whether the difference between the cuff inflation parameter value determined during the corresponding inflation operation and the cuff inflation parameter value determined during a preceding inflation operation meets a predetermined criterion.

### FIELD OF INVENTION

The invention is generally directed at a non-invasive blood pressure measurement system. More particularly, various inventive methods and apparatus disclosed herein relate a detection device for verifying whether an inflatable cuff of the non-invasive blood pressure measurement system is wrapped around a measurement site of a patient and connected to the non-invasive blood pressure measurement system.

### BACKGROUND OF THE INVENTION

High blood pressure is a significant risk factor for heart attack, stroke, kidney disease, and vision loss. It often is referred to as the silent killer because it rarely causes any symptoms until considerable organ damage has occurred. For that reason, obtaining regular, accurate blood pressure readings are important to long-term health. In hospital and ambulatory setting, constant or periodic blood pressure monitoring is essential component of monitoring patient's vital signs.

For many years blood pressure has been measured using an upper arm pressure cuff and auscultation of the brachial artery to identify the appearance and disappearance of Korotkoff sounds. Although the auscultatory method employing mercury sphygmomanometers used to be regarded as the “gold standard” for blood pressure measurement, widespread implementation of the ban in use of mercury sphygmomanometers continues to diminish the role of this technique. When home monitoring was first introduced, most approached relied upon aneroid sphygmomanometers.

Increasingly, automated devices for measuring blood pressure are now used in the clinic, hospitals and by people in their homes. In addition, ambulatory blood pressure measurement devices are available that are programmed to allow blood pressure to be measured repeatedly during the day and night. The standard type of monitor for home or ambulatory use is now an oscillometric device that records pressure from the brachial artery. These have the advantage of being easy to use, because cuff placement is not as critical as with devices that use a Korotkoff sound microphone, and the oscillometric method has in practice been found to be as reliable as the Korotkoff sound method.

Nowadays, automated blood pressure monitoring has rapidly essentially replaced the traditional mercury or aneroid sphygmomanometers and the stethoscope, becoming an essential tool of patient care and home healthcare.

Conventional non-invasive blood pressure (“NIBP”) measurement systems engage the sphygmo-manometric occlusive limb-cuffs that are applied around an extremity of a patient's body, e.g. wrapped around a patient's upper arm. When the NIBP system is used, the blood pressure cuff is placed around a limb of a patient and is inflated to an initial inflation pressure that fully occludes the brachial artery to temporarily prevent blood pressure flow. The cuff is then deflated from the initial inflation pressure and the pressure transducer detects pressure pulses associated with the patient's heartbeat, as blood begins to flow past the pressure cuff. Alternatively, the cuff is inflated slowly until the brachial artery is occluded completely, the pressure transducer detects pressure pulses associated with the patient's heartbeat during inflation and then the cuff is deflated rapidly.

Known NIBP systems support various cuff sizes, from neonatal to extra-large adult cuffs, where the inflation timeout has to be long enough to allow the inflation of the largest supported cuff loosely wrapped around the largest supported limb, because the NIBP system usually does not recognize which size cuff is actually connected. Consequently, the timeout makes it unsuitable for detecting whether or not the cuff is wrapped around a limb for smaller cuffs because a smaller cuff can be inflated to the target pressure faster than the timeout period even though it is not wrapped around a limb.

Not being able to detect whether the cuff is wrapped around a patient's limb (“cuff off” detection) is particularly detrimental to automatic blood pressure monitoring systems capable of programmable measurement sequences, because a clinical staff or a patient may intentionally remove or accidentally dislodge the cuff from the measurement site on the limb or disconnect the cuff from the NIBP monitor without stopping the automatic measurement mode or programmable measurement sequence. As a result, the NIBP system continues to take measurements and may obtain phantom readings due to cuff pressure oscillations caused by stretching of the cuff or movement of the cuff induced by external vibrations, etc.

U.S. Pat. No. 8,808,189B2 discloses a method of detecting the wrapping strength of a cuff that is wrapped around the limb and not detached from it. The detection is made after the pressure-volume relationship according to a change of the cuff pressure detected in the cuff wrapped around the measurement site and, equally, volume change of the cuff detected by the volume detection unit with the change of its pressure whilst it is controlled by pressurization or depressurization by the pressure control unit. However, this approach does not inform whether or not the inflatable cuff is attached to the measurement site of a patient. U.S. Pat. No. 4,669,485A discloses apparatus and related methods for continuous long term non-invasive measurement of the pressure of a pulsatile fluid flowing through a flexible tube, particularly human arterial blood flow, is disclosed. Specifically, the apparatus provides a continuous calibrated pressure measurement by first undertaking a “calibration” phase comprised of determining the pressure at various pre-defined conditions of flow and, in response thereto, ascertaining the values of a plurality of coefficients each of which is associated with a corresponding term in a pre-defined function that characterizes fluid pressure in relation to pulsatile displacement of the wall of the tube; and second, undertaking a “continuous monitoring” phase comprised of determining each subsequently occurring pressure value as the pre-defined function of each corresponding pulsatile wall displacement value, and re-initiating the calibration phase at the expiration of pre-defined time intervals which adaptively change based upon current and prior results.

### SUMMARY OF THE INVENTION

Generally, it is an object of the present invention to enable cuff-off detection for blood pressure measurement devices. More particularly, in its various embodiments, the invention focuses on a blood pressure measurement system and apparatus capable of detecting improper functioning due to the absence of an inflatable cuff from a patient's measurement site, its improper attachment to the site, or it being disconnected from the blood pressure measurement system.

According to the first aspect of the invention, this object is addressed by a detection device suitable for use in a blood pressure measurement system, wherein said device is coupled to an inflatable cuff attached to a measurement site at a patient's body part. The device is arranged to perform a series of blood pressure measurements, wherein for each measurement of the series, an inflation operation is performed to inflate the cuff. The device includes a determining unit for determining inflation speed-dependent parameter values during inflation operations, and a processor module arranged for receiving the determined inflation speed-dependent parameter values. The processor module is configured to check whether the difference between the cuff inflation parameter value determined during the corresponding inflation operation and the cuff inflation parameter value determined during a preceding inflation operation meets a predetermined criterion. As explained above, it is advantageous to detect whether a cuff is disconnected from a blood pressure measurement system or unattached to the patient's limb to avoid phantom readings during a series of automatic measurements. If the inflation parameter value determined during a preceding inflation operation does not meet a predetermined criterion, the measurements are immediately stopped.

In various embodiments, the corresponding inflation operation and the preceding inflation operation are sequential operations in an automatic measurement series and/or programmable measurement sequences. The “cuff off” detection is particularly important during a series of automatic measurements and programmable measurement sequences.

In various embodiments, the inflation speed-dependent parameter is defined as a period of time required during the inflation operation to bring the pressure of the cuff from the first pressure level to the second pressure level.

In some embodiments, the first pressure level is a pressure of the cuff at the start of the inflation operation. The second pressure level may be any pressure between the first pressure level and the target cuff pressure at the end of the inflation operation. Preferably, the second pressure level is substantially smaller than the target cuff pressure at the end of the inflation operation in order to stop an unnecessary inflation as soon as possible. In many embodiments, the inflation speed-dependent parameter is the first derivative (dP/dt) of the cuff pressure during inflation.

In various embodiments, the device is arranged to stop the inflation process if the difference between the measured inflation speed-dependent parameter and the reference inflation-speed dependent parameter exceeds a predetermined value. All measurements are conducted during the inflation process, which helps to both avoid running the pump for a long time (more than 1 minute) before timeout occurs or deformation of the cuff due to overlong inflation.

According to the second aspect of the invention, the invention focuses on a method for checking whether an inflatable cuff has been disconnected from the device or has been attached to a measurement site of a body part of a patient. The method is performed by a device for using in a blood pressure measurement system, which is coupled to the inflatable cuff attached to a measurement site of a body part of a patient. The device performs a series of blood pressure measurements, whereby for each measurement of the series an inflation operation is performed to inflate the cuff. The method includes the following steps: determining an inflation speed-dependent parameter value during the inflation operation, receiving the inflation speed-dependent parameter values determined, and determining the difference between the cuff inflation parameter value determined during the corresponding inflation operation and the cuff inflation parameter value determined during a preceding inflation operation.

It should be appreciated that all combinations of the previous concepts and the additional concepts discussed in greater detail below (provided such concepts are not mutually inconsistent) are contemplated as being part of the inventive patient matter disclosed herein. In particular, all combinations of the claimed patient matter appearing at the end of this disclosure are contemplated as being part of the inventive patient matter disclosed herein. It should also be appreciated that the terminology explicitly employed herein also appearing in any disclosure incorporated by reference should be accorded a meaning most consistent with the particular concepts disclosed herein.

### BRIEF DESCRIPTION OF THE DRAWINGS

In the drawings, similar reference characters generally refer to the same parts throughout different views. Also, the drawings are not necessarily to scale, with the emphasis instead generally being placed upon illustrating the principles of the invention.

FIG. 1 shows a block diagram of a system for non-invasive of monitoring blood pressure with the device for checking whether an inflatable cuff is attached to a patient's limb;

FIG. 2 shows a graph of the cuff inflation and deflation cycle;

FIG. 3 shows a graph of the cuff inflation process; and

FIG. 4 shows the method's flow diagram;

### DETAILED DESCRIPTION OF THE INVENTION

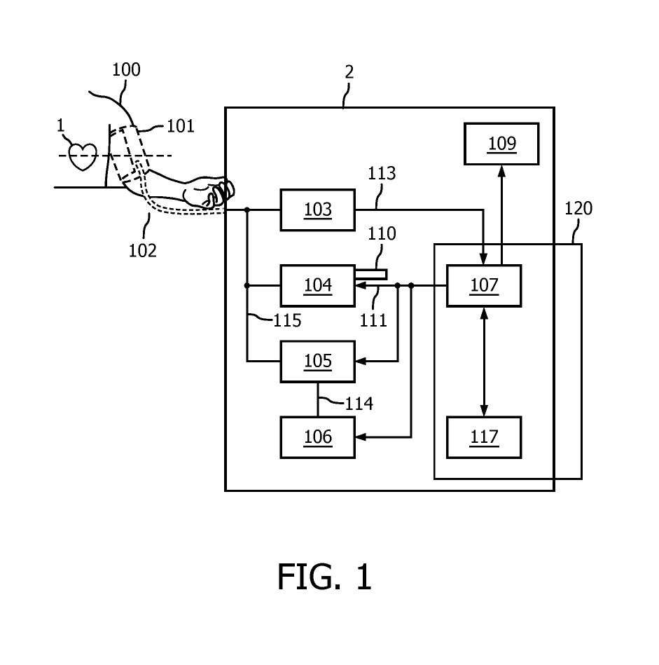

Referring to FIG. 1, in one embodiment of the invention, a non-invasive blood pressure monitoring system includes an inflatable cuff 101 connected to the blood pressure monitoring system and attached to the measurement site; for example, an upper arm 100 of a patient 1. The system includes a display 109 for displaying measurement results, a main processor module 107, a memory unit 117 and an air system including: a source of pressured air 106, an optional inflate valve 105, deflate valve(s) 104, and pressure transducer(s) 103.

The blood pressure cuff 101 is connected by a hose 102 to the housing of system 2 and can be inflated and deflated for occluding the brachial artery of the patient 1 when in the fully inflated condition. As the blood pressure cuff 101 is deflated using deflate valves(s) 104 via an exhaust 110, the arterial occlusion is gradually relieved. The deflation of the blood pressure cuff 101 by the deflate valve(s) 104 is controlled by the central processor module 107 through a control line 111.

A pressure transducer 103 is coupled via the hose 102 to the blood pressure cuff 101 for sensing the pressure within the cuff 101. In accordance with conventional oscillometric techniques, the pressure transducer 103 is used to sense pressure oscillations in the cuff 101 that are generated by pressure changes in the artery under the cuff. The electrical oscillation signals from the pressure transducer 103 are obtained by the central processor module 107, using an analog to digital converter through a connection line 113.

The source of compressed air 106 comprises a pump or gas cylinder filled with compressed air. The compressed air is supplying the pressured air via duct 114 to the inflate valve(s) 105. The operation of the inflate valve(s) 105 or source of pressurized air 106 is controlled by the central processor module 107 through the control line 111. Thus, the inflation and deflation of the blood pressure cuff 101 is controlled by the central processor 107 through the deflate valve(s) 104 and the inflate valve(s) (105) or source of pressurized air 106, respectively.

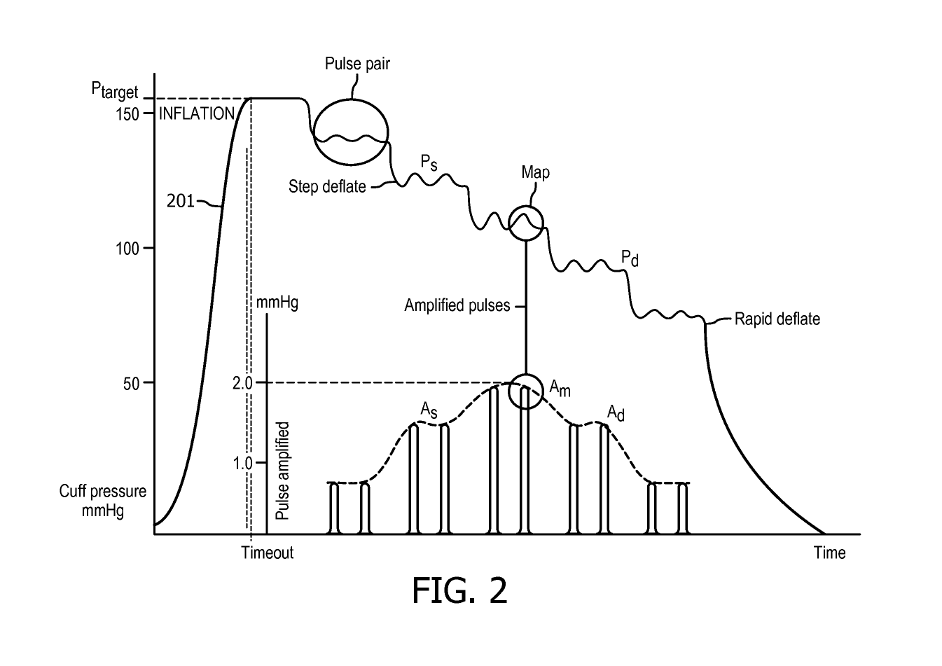

FIG. 2 illustrates the measurement cycle for oscillometric blood pressure. The oscillometric method measures blood pressure by monitoring the pulsatile changes in pressure that are caused by the flow of blood through an artery that is restricted by an occluding cuff. The cuff pressure for a measurement cycle, as measured by the transducer, is characterized by the wave 201. The cuff pressure rapidly increases to a maximum above the patient's systolic Pₛ pressure and is then deflated in a sequence of steps to a point below the diastolic pressure P_{d}. As the pressure in the cuff decreases, blood begins to flow through the artery. The sensitive transducer measures cuff pressure and small pressure oscillations within the cuff. A typical determination takes 10-12 deflation steps. Each step is made sufficiently long enough to include at least one heartbeat. As the pressure in the cuff decreases further, the pulses reach maximum amplitude Aₘ. The pressure in the cuff that corresponds to the point of the maximum oscillation has been shown to correlate to a patient's mean arterial pressure (MAP). As the pressure in the cuff is decreased further, the pulses begin to decrease in amplitude (A_{d}). The rising and falling amplitude of the pressure pulses creates an envelope that is used to determine the patient's systolic (Pₛ), mean (Pₘ) and diastolic (P_{d}) pressures. The algorithm determines a patient's systolic and diastolic pressures by locating the points on the pulse pressure envelope that correspond to a predetermined percentage of the maximum amplitude (Aₘ). NIBP measurements can be taken manually, i.e. when each time only one measurement is taken, and automatically, when the measurement is repeated at specified intervals. If an automatic measurement series or programmable measurement series is running, the NIBP device according to various embodiments of the present invention measures the duration from the start of the inflation until a predefined cuff pressure level is reached (P_{target}). If this duration has increased significantly from the previous to the current inflation, the NIBP monitor indicates that the cuff has been disconnected from the device or is no longer applied to the patient's limb, aborts the current inflation, stops the automatic measurement series or programmable measurement sequence and issues a technical alarm; e.g. “Check Cuff”.

Furthermore, the verification of whether or not an inflatable cuff is attached to the measurement site of a patient is performed during the inflation process. When performing the verification, the processor 107 checks whether the difference between the cuff inflation parameter value determined during the corresponding inflation operation and the cuff inflation parameter value determined during a preceding inflation operation meets a predetermined criterion. If the predetermined conditions are met, the measurements continue and, if not, the measurements terminate.

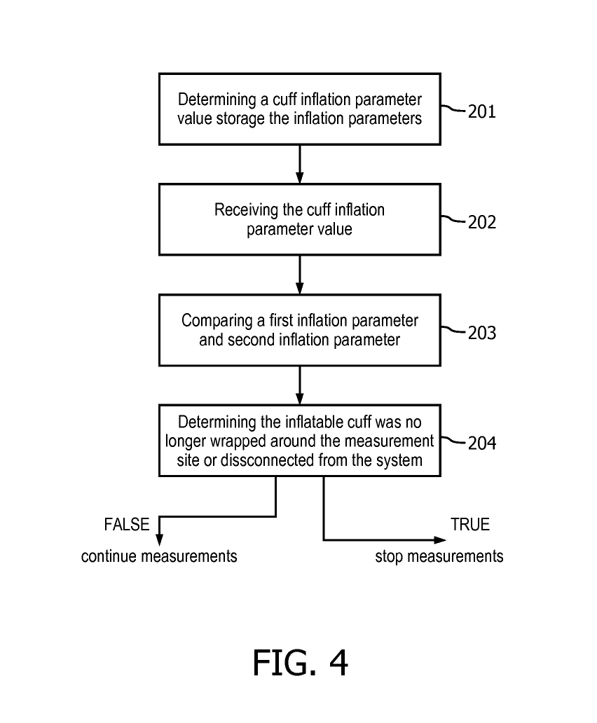

FIG. 4 shows a diagram illustrating a method, performed by the blood pressure monitoring system 2. In the first step 201, the cuff inflation parameter values are determined in the processor 107 and stored in the memory unit 117. In the next step 202, the processor 107 reads the cuff inflation parameter values determined during two corresponding inflation operations. The processor 107 also compares the first inflation parameter with the second one in step 203. Based on the result of the comparison, the processor 107 decides in step 204 whether to continue or stop the measurements.

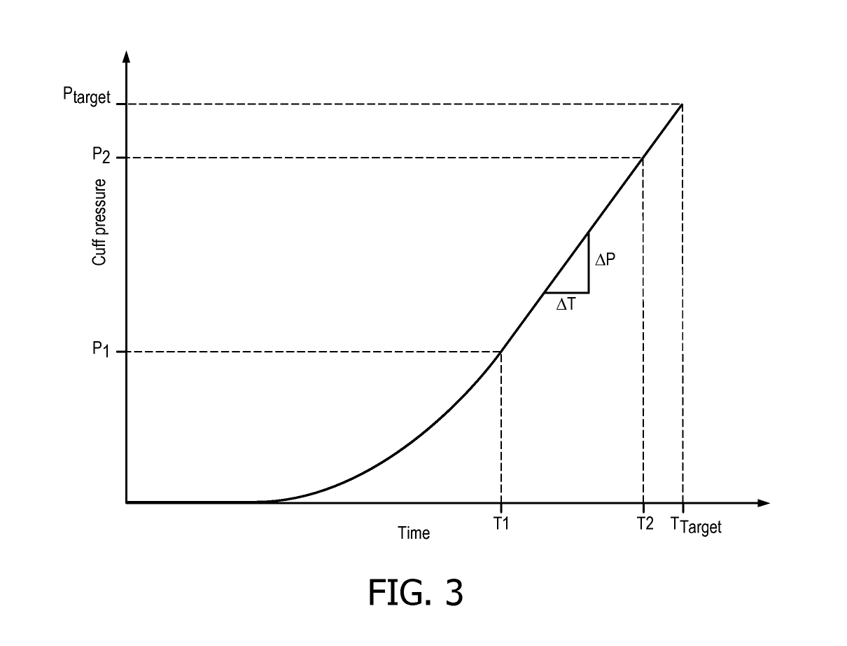

Referring again to FIG. 2, cuff pressure dependency over time during a blood pressure measurement operation is shown. The graph illustrates the time available for the inflation to a target cuff pressure P_{target}, called timeout, which should be long enough to allow the inflation of the largest supported cuff loosely wrapped around the largest supported limb to the highest cuff pressure. The timeout is dependent on some variables, such as cuff size, the power of the source of pressured air, how tight the cuff is wrapped around a limb, the target cuff pressure, etc. Since it is not desirable to run a source of pressured air 106 without stopping, a timeout is set as a kind of safety mechanism that stops the source of pressured air 106 after the maximum expected time is over. Consequently, the timeout is not suitable to detect whether the cuff 101 is wrapped around a limb for smaller cuffs 101 because a smaller cuff 101 can be inflated to the target pressure even when it not wrapped around a limb. In the non-invasive blood pressure monitoring system 2 shown in FIG. 1, a subsystem 120, comprising the processor 107 and the memory unit 117, measures the speed-dependent parameter when an automatic measurement series or programmable measurement series is running. As a speed-dependent parameter, a period of time required during the inflation operation that brings the pressure of the cuff 101 from the first level (P1) to the second level (P2) (see FIG. 3) can be used. In a first embodiment, the first pressure level is slightly above the pressure of the cuff 101 when an inflation process starts (t=0), and the second pressure level is any pressure between the first pressure level and the target cuff pressure P_{target} at the end of the inflation operation. Preferably, the second pressure level is substantially smaller than the target cuff pressure P_{target} at the end of the inflation operation, such that an unnecessary inflation stops as soon as possible if the cuff is dislodged from the desired measurement site. This means that the second pressure level is selected such a patient feels no or only limited discomfort in his or her arm until the inflation process stops, if the cuff is accidentally or intentionally moved from the intended measurement site. In another embodiment, the inflation speed-dependent parameter is the first derivative dP/dt of the cuff pressure during inflation.

The processor 107 receives the inflation speed-dependent parameters and when a blood pressure monitoring system is in the automatic measurement series or programmable measurement series mode, the processor checks the difference between the cuff inflation parameter values determined during corresponding inflation operations in the measurement series. The processor 107 stops the inflation operation if the difference between the inflation speed-dependent parameter of the corresponding and the previous inflation operations exceeds a predetermined criterion.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

It should also be understood that, unless clearly indicated to the contrary, in any methods claimed herein that include more than one step or act, the order of the steps or acts of the method is not necessarily limited to the order in which the steps or acts of the method are recited. Also, reference numerals appearing in the claims in parentheses pursuant to Rule 6.2(b) of the Patent Cooperation Treaty (“PCT”), are provided merely for convenience and should not be viewed as limiting in any way.

1. A detection device suitable for use in a blood pressure measurement system, said device being configured for coupling to an inflatable cuff attached to a measurement site of a body part of a patient and arranged to perform a series of blood pressure measurements, wherein, for each measurement of the series, an inflation operation is performed to inflate the cuff, the device comprising:
a determining unit for determining inflation speed-dependent parameter value during each inflation operation of the series,
a memory unit arranged to store the determined inflation speed-dependent parameter value; and
a processor module arranged for receiving the determined inflation speed-dependent parameter values from the memory unit; and for checking whether a difference between inflation speed-dependent parameter values determined during a first inflation operation and during a second inflation operation meets a predetermined criterion, wherein in the series of blood pressure measurements the second inflation operation is subsequent to the first inflation operation; and
wherein the processor module is further arranged to stop the inflation operation if the difference between the inflation speed-dependent parameter values exceeds the predetermined criterion.

2. The device of claim 1, wherein the second inflation operation immediately follows the first inflation operation in the series of blood pressure measurements.

3. The device of claim 1, wherein the inflation speed-dependent parameter is defined as a period of time required during the inflation operation to bring the pressure of the cuff from the first pressure level to the second pressure level.

4. The device of claim 1, wherein the first pressure level is a pressure of the cuff at a start of the inflation operation.

5. The device of claim 1, wherein the second pressure level is a pressure level between the first pressure level and a target cuff pressure level at the end of the inflation operation.

6. The device of claim 5, wherein the second pressure level is substantially smaller than the target cuff pressure at the end of the inflation operation.

7. (canceled)

8. The device of claim 7, wherein the memory unit is further arranged for storing reference speed-dependent parameters for different cuff sizes and wherein the device is arranged to compare the measured inflation speed-dependent parameter with a selected one of the reference inflation-speed dependent parameters stored in the memory.

9. The device of claim 1, wherein an inflation speed-dependent parameter is the first derivative (dP/dt) of the cuff pressure during inflation.

10. The device of claim 1, wherein the device is further arranged to stop the inflation process if the difference between the measured inflation speed-dependent parameter and a reference inflation-speed dependent parameter exceeds a predetermined value.

11. A blood pressure measurement system comprising the device according to claim 1, the inflatable cuff and a hose, wherein the device is coupled to the cuff via the hose.

12. A method of checking whether an inflatable cuff has been disconnected from the device or has been attached to a measurement site of a body part of a patient, the method being performed by a device suitable for use in a blood pressure measurement system, coupled to the inflatable cuff attached to a measurement site of a body part of a patient, wherein the device performs a series of blood pressure measurements, wherein for each measurement of the series an inflation operation is performed to inflate the cuff, the method comprising the steps of:
determining an inflation speed-dependent parameter value during each inflation operation of the series,
storing the determined inflation speed-dependent parameter value in a memory unit;
receiving the determined inflation speed-dependent parameter values from the memory unit, and
determining a difference between an inflation speed-dependent parameter value determined during the a first inflation operation and a second inflation operation, wherein in the series of blood pressure measurements the second inflation operation is subsequent to the first inflation operation; and wherein the method further comprises
stopping the inflation operation if the difference between the inflation speed-dependent parameter value exceeds the predetermined criterion.
